(19) United States
(12) Patent Application Publication
(10) Pub. No.: US 2018/0116592 A1
(43) Pub. Date: May 3, 2018

(54) FIBROUS TRANSISTOR AND METHOD FOR MANUFACTURING THE SAME

(71) Applicant: KOREA INSTITUTE OF SCIENCE AND TECHNOLOGY, Seoul (KR)

(72) Inventors: Jung Ah LIM, Seoul (KR); SOO JIN KIM, Seoul (KR); Jeon Kook LEE, Seoul (KR); Do Kyung Hwang, Seoul (KR)

(21) Appl. No.: 15/429,196

(22) Filed: Feb. 10, 2017

(30) Foreign Application Priority Data
Nov. 3, 2016 (KR) 10-2016-0145604

(51) Int. Cl. A61B 5/00; H01L 29/786; H01L 51/05; A61B 5/0492; G01N 27/414

(52) U.S. Cl. CPC A61B 5/6804; H01L 29/7869; H01L 51/0512; A61B 2562/125; G01N 27/4145; A61B 2560/0468; A61B 2562/0209; A61B 5/0492

(57) ABSTRACT

The present invention relates to a fibrous transistor, and a method of manufacturing the same, and more particularly, to a fibrous transistor, in which a source fiber and a drain fiber are formed in a twisted state in a longitudinal direction, so that a contact surface of the source fiber and the drain fiber is increased, thereby enabling charges to easily move, and a method of manufacturing the same. Further, the present invention relates to a fibrous transistor, in which a gate insulating layer is formed by using ion gel, so that the fibrous transistor may obtain a high current at the same operation voltage, thereby having a low operation voltage, and a method of manufacturing the same.

### CROSS-REFERENCE TO RELATED APPLICATIONS

This application claims priority to and the benefit of Korean Patent Application No. 10-2016-0145604 filed in the Korean Intellectual Property Office on Nov. 3, 2016, the entire contents of which are incorporated herein by reference.

### TECHNICAL FIELD

The present invention relates to a fibrous transistor, and a method of manufacturing the same, and more particularly, to a fibrous transistor, in which a source fiber and a drain fiber are formed in a twisted state in a longitudinal direction, so that a contact surface of the source fiber and the drain fiber is increased, thereby enabling charges to easily move, and a method of manufacturing the same.

The present invention relates to a fibrous transistor, in which a gate insulating layer is formed by using ion gel, so that the fibrous transistor may obtain a high current at the same operation voltage, thereby having a low operation voltage, and a method of manufacturing the same.

### BACKGROUND ART

Recently, as a wearable electronic device has been settled as a paradigm of the age, research on an electronic textile, in which a textile, such as clothes, is combined with a function of an electronic device, is actively conducted. Since the textile is flexible and comfort, even when a person wears the textile all day long, the person feels minimal fatigue, so that the textile attracts attentions as an ideal platform of a wearable electronic device.

The existing wearable electronic device is stayed in the form, in which a hard solid electronic device or sensor is simply attached onto cloth, or devices are connected by using a conductive fiber, so that it is impossible to expect comfort of the textile. In order to solve the problem, a development of an electronic device, which is insertable into cloth having a form of yarn, which is capable of maintaining a characteristic of a textile, is required.

Among the various electronic devices, a transistor is a basic switching device in driving an electronic device, such as a sensor and a display, and is an essential component in implementing a fibrous electronic device.

A generally used gate insulator of a transistor is metal oxides, such as SiO₂ or Al₂O₃. The field effect transistor (FET) has an advantage in that a switching speed is high, but the FET requires a vacuum process in a manufacturing process, so that operation cost and capital expenses are high. Further, there is a problem in that the metal oxide has a high driving voltage due to low capacitance.

When the gate insulator of the transistor is replaced with an electrolyte, the transistor has a relatively low switching speed, but the transistor may obtain a higher charge carrier density due to a high electric capacity of the electrolyte, so that the transistor is actively researched in an electronic technology field requiring a low operation voltage and a high output current. The electrochemical transistor, which uses an electrolyte as a gate insulator, uses an electrolyte, such as a polymer electrolyte based on an ionic liquid, as an insulator, so that the electrochemical transistor is drivable at a low voltage and has the very low quantity of power consumption, thereby advantageously applied to a portable device.

However, it is difficult to precisely adjust an arrangement of three electrodes including source/drain/gate electrodes, in a flat transistor in the related art, so that there is a problem in implementing a fibrous transistor. Further, the flat transistor in the related art has a high driving voltage, so that the flat transistor cannot actually be applied to a wearable product.

### SUMMARY OF THE INVENTION

The present invention has been made in an effort to provide a fibrous transistor, in which an ion gel having high ionic conductivity surrounds an external side of a source fiber and a drain fiber, which are twisted in a longitudinal direction, in order to decrease a driving voltage and remove a limitation of a position, at which a gate may be arranged.

The present invention has also been made in an effort to provide a fibrous transistor, in which a source fiber and a drain fiber are twisted in a longitudinal direction in order to increase a movement passage of charges generated in the source fiber.

An exemplary embodiment of the present invention provides a fibrous transistor, including: a source fiber; a drain fiber; an ion gel layer provided so as to surround the source fiber and the drain fiber; and a gate positioned at a part of an external side of the ion gel layer, in which the source fiber and the drain fiber form one or more contact surfaces in a twisted state.

The source fiber may include a conductive fiber, and a semiconductor layer formed on an outer circumferential surface of the conductive fiber.

The drain fiber may include a conductive fiber, and a semiconductor layer formed on an outer circumferential surface of the conductive fiber.

The source fiber and the drain fiber may be alternately twisted with each other in a longitudinal direction.

In a longitudinal direction of any one fiber between the source fiber and the drain fiber, the other fiber may be wound around the one fiber in a spiral form.

The gate may be wound in a spiral form in a longitudinal direction of the ion gel layer.

The conductive fiber may include a conductive material.

The semiconductor layer may include one or more of an organic semiconductor, an oxide semiconductor, a metal oxide semiconductor, and a carbon compound semiconductor.

The ion gel layer may include at least one of 1-ethyl-3-methylimidazolium bis(trifluoromethylsulfonyl)imide ([EMIM][TFSI]), poly(vinyl phosphonic acid-co-acrylic acid) (P(VPA-AA)), poly(styrene sulfonic acid) (PSSH), NaCl among the poly ethylene oxide (PEO) matrixes, polyvinylidenefluoride (PVDF), 1-butyl-3-methylimidazolium bis (trifluoromethanesulfonimide) ([bmim][Tf2N]), polymer IL poly(l-vinyl-3-methylimidazolium bis(tri-fluoromethanesulfonimide) (poly[ViEtIm][Tf2N]), PEO/LiTFSI, 1-butyl-3-methylimidazolium hexafluorophosphate ([BMIM][PF6]), and 1-ethyl-3-methylimidazolium n-octylsulfate ([EMIM][OctOSO3]).

Another exemplary embodiment of the present invention provides a method of manufacturing a fibrous transistor, the method including: (a) manufacturing a source fiber; (b) manufacturing a drain fiber; (c) twisting the source fiber and the drain fiber so as to form one or more contact surfaces; (d) forming an ion gel layer so as to surround the twisted source fiber and drain fiber; and (e) positioning a gate at a part of an external side of the ion gel layer.

Operations (a) and (b) may further include: preparing a conductive fiber; and forming a semiconductor layer on an outer circumferential surface of the conductive fiber.

In operation (c), the source fiber and the drain fiber may be alternately twisted with each other in a longitudinal direction.

In operation (c), in a longitudinal direction of any one fiber between the source fiber and the drain fiber, the other fiber may be wound around the one fiber in a spiral form.

In operation (d), the ion gel layer may be formed by any one method of aerosol jet printing, ink jet printing, transfer printing, spin coating, dip coating, and casting.

In operation (e), the gate may be positioned in any one form among a spiral form, a parallel form, and a twist form in a longitudinal direction of the ion gel layer.

Yet another exemplary embodiment of the present invention provides an electromyogram sensor including a transistor manufactured by the method of manufacturing the fibrous transistor.

Yet another exemplary embodiment of the present invention provides an electrocardiogram sensor including a transistor manufactured by the method of manufacturing the fibrous transistor.

According to the present invention, the ion gel layer is provided so as to surround the source fiber and the drain fiber, so that it is possible to flexibly dispose a gate electrode without being limited to a lamination structure of a semiconductor/gate dielectric substance/gate electrode.

The ion gel layer serves as a gate insulating layer, so that the fibrous transistor may be operated at a low voltage of several voltages or less, thereby increasing electric capacitance.

The source fiber and the drain fiber are formed in a twisted state, so that a contact surface of the source fiber and the drain fiber is increased, and the quantity of current of the fibrous transistor is increased.

### BRIEF DESCRIPTION OF THE DRAWINGS

FIG. 1 is a perspective view of a fibrous transistor according to an exemplary embodiment of the present invention.

FIG. 2 is a perspective view of a fibrous transistor according to another exemplary embodiment of the present invention.

FIG. 3 is a cross-sectional view of the fibrous transistor according to the exemplary embodiment of the present invention.

FIG. 4 is a perspective view illustrating a method of manufacturing a fibrous transistor according to an exemplary embodiment of the present invention.

FIG. 5 is a perspective view illustrating a method of manufacturing a source fiber and a drain fiber according to an exemplary embodiment of the present invention.

FIG. 6 is a flowchart illustrating a method of manufacturing a fibrous transistor according to an exemplary embodiment of the present invention.

FIG. 7 is a picture of a fibrous transistor according to a manufacturing example.

FIG. 8 is a graph representing driving performance of the fibrous transistor according to the present invention.

FIG. 9 is a graph representing a change in a drain current according to a change in a gate voltage of the fibrous transistor according to the present invention.

### DETAILED DESCRIPTION

The present invention will be described in detail below with reference to the accompanying drawings. Herein, the repeated description, the detailed description of a known function and configuration that may make the purpose of the present invention unnecessarily ambiguous will be omitted. Exemplary embodiments of the present invention are provided so as to completely explain the present invention to those skilled in the art. Accordingly, the shape, the size, etc., of elements in the figures may be exaggerated for explicit comprehension.

Throughout the specification and the claims, unless explicitly described to the contrary, the word “include/comprise” and variations such as “includes/comprises” or “including/comprising” mean further including other constituent elements, not excluding the other constituent elements.

Hereinafter, exemplary embodiments will be provided for helping understanding of the present invention. However, the following exemplary embodiments are provided only for the purpose of making the present invention be more easily understood, and thus the contents of present invention are not limited by the exemplary embodiment.

<Fibrous Transistor>

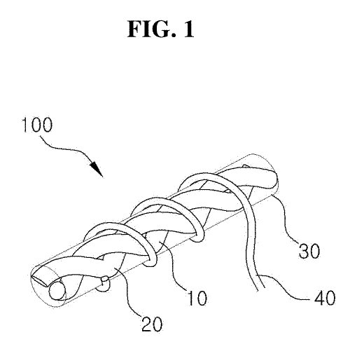

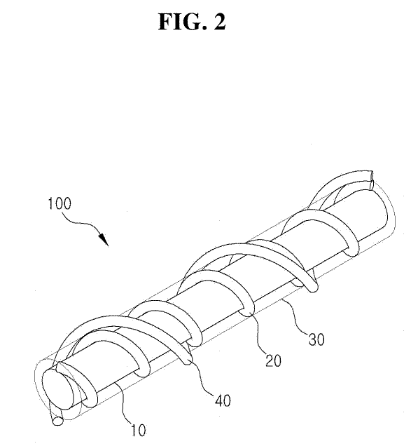

FIG. 1 is a perspective view of a fibrous transistor 100 according to an exemplary embodiment of the present invention. FIG. 2 is a perspective view of a fibrous transistor 100′ according to another exemplary embodiment of the present invention. Referring to FIGS. 1 and 2, each of the fibrous transistors 100 and 100′ according to the exemplary embodiments of the present invention includes a source fiber 10, a drain fiber 20, an ion gel layer 30 provided so as to surround the source fiber 10 and the drain fiber 20, and a gate 40 positioned at a part of an exterior side of the ion gel layer 30.

The source fiber 10 and the drain fiber 20 may form one or more contact surfaces in a twisted state. For example, the source fiber 10 and the drain fiber 20 may form a twisted shape while crossing in the longitudinal direction (see FIG. 1), or in a longitudinal direction of any one of the source fiber 10 and the drain fiber 20, the other fiber may be wound around the one fiber in a spiral shape (see FIG. 2).

The case where the source fiber 10 and the drain fiber 20 are twisted in a spiral shape includes two types. First, when the source fiber 10 serves as the basis, the drain fiber 20 may be wound in the longitudinal direction of the source fiber 10 in a spiral shape. Next, when the drain fiber 20 serves as the basis, the source fiber 10 may be wound in the longitudinal direction of the drain fiber 20 in a spiral shape.

Here, the source fiber 10 may supply charge carriers, and the drain fiber 20 may receive charge carriers. The source fiber 10 may include a conductive fiber 11, and a semiconductor layer 12 formed on an outer circumferential surface of the conductive fiber 11. Further, the drain fiber 20 may include a conductive fiber 21, and a semiconductor layer 22 formed on an outer circumferential surface of the conductive fiber 21. The semiconductor layers 12 and 22 may serve as movement passages, through which charges generated in the conductive fiber 11 included in the source fiber 10 are movable to the conductive fiber 21 included in the drain fiber 20.

The conductive fibers 11 and 21 may be formed of a conductive material, and a metal or a conductive polymer material may be used as the conductive material, and the conductive material may include any one of gold (Au), silver (Ag), aluminum (Al), copper (Cu), nickel (Ni), molybdenum (Mo), tungsten (W), an indium tin oxide (ITO), polythiophene, polyaniline, polyacetylene, olypyrrole, poly phenylene vinylene, a mixture of polyethylenedioxythiophene (PEDOT)/polystyrenesulfonate (PPS), a carbon fiber, and a carbon nano tube (CNT).

For example, in the case where the source fiber 10 and the drain fiber 20 are formed by using a carbon nano tube, the carbon nano tube is a static electricity discharging material, so that the carbon nano tube may have an effect in that when the source fiber 10 and the drain fiber 20 are twisted with each other, the charges accumulated in the source fiber 10 and the drain fiber 20 are momentarily discharged and static electricity is generated, and bonding force between the source fiber 10 and the drain fiber 20 is increased.

However, as long as the conductive fibers 11 and 21 have conductivity, the materials of the conductive fibers 11 and 21 are not particularly limited.

The semiconductor layers 12 and 22 may include one or more of an organic semiconductor, an oxide semiconductor, a metal oxide semiconductor, and a carbon compound semiconductor. The organic semiconductor may be selected from a monomolecule selected from a pentacene-based material, a benzothiophene-based material including C8-BTBT(2,7-dioctyl[1]ben-zo-thieno[3,2-b][1] benzothiophene, and diF-TESADT(2,8-difluoro-5,11-bis(triethylsilylethynyl)anthradithiophene), polyphenylene vinylene, thiophene vinylene, P3HT(poly(3-hexylthiophene), PBTTT (poly(2,5-bis(3-alkylthiophen-2-yl) thieno-[3,2-b]thiophene), cyclopentadithiophene (CPDT), indacenodithiophene (IDT), benzodithiophene (BDT), benzotrithiophene (BTT), naphthalene diimide (NDI), polythiophenes, and polyfluorenes, and the oxide semiconductor may be formed of a ternary system or quaternary system oxide semiconductor formed of a combination of AxByCzO (A, B, C=Zn, Cd, Ga, In, Sn, Hf, Zr; x, y, z≥0). Further, the oxide semiconductor may be amorphous or crystalline, or a crystal structure, in which an amorphous material and a crystalline material are mixed.

The metal oxide semiconductor may use any one selected from a zinc oxide (ZnOx), an indium oxide (InOx), an indium gallium zinc oxide (IGZO), and an indium tin oxide (ITO), and the carbon compound semiconductor may use a semiconductor carbon nano tube (CNT) or a graphene nano ribbon.

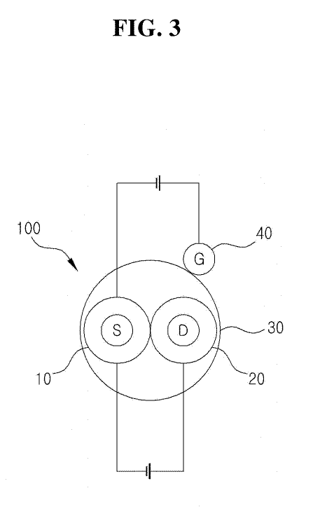

FIG. 3 is a cross-sectional view of the fibrous transistors 100 and 100′ according to the exemplary embodiments of the present invention. An operation principle of the fibrous transistors 100 and 100′ will be described with reference to FIG. 3. Charges are generated in the source fiber 10 by a voltage applied to the source fiber 10 and the gate 40, and the charges generated in the source fiber 10 move to the drain fiber 20, so that a current flows in the fibrous transistors 100 and 100′. In this case, the source fiber 10 and the drain fiber 20 are formed in the twisted state, so that a contact surface serving as a movement passage, through which the charges are movable, is increased, and the quantity of currents of the fibrous transistors 100 and 100′ is increased.

The ion gel layer 30 may generally serves as a gate insulating layer. The ion gel layer 30 is a configuration for insulating the source fiber 10 and the drain fiber 20 from the 40, and may be formed by hardening an ionic liquid. In order to apply the ionic liquid to the electronic device and use the ionic liquid, a method of assigning a mechanical property by adding a material forming a 3D structure within the ionic liquid without hindering the movement of the ion as possible as it can is required. An inorganic material or a polymer material may be used as the material of forming the 3D structure.

The ion gel layer 30 uses an ionic liquid using a polymer material, and may include at least one of 1-ethyl-3-methylimidazolium bis(trifluoromethylsulfonyl)imide ([EMIM][TFSI]), poly(vinyl phosphonic acid-co-acrylic acid) (P(VPA-AA)), poly(styrene sulfonic acid) (PSSH), NaCl among the poly ethylene oxide (PEO) matrixes, polyvinylidenefluoride (PVDF), 1-butyl-3-methylimidazolium bis (trifluoromethanesulfonimide) ([bmim][Tf2N]), polymer IL poly(l-vinyl-3-methylimidazolium bis(tri-fluoromethanesulfonimide) (poly[ViEtIm][Tf2N]), PEO/LiTFSI, 1-butyl-3-methylimidazolium hexafluorophosphate ([BMIM][PF6]), and 1-ethyl-3-methylimidazolium n-octylsulfate ([EMIM][OctOSO3]).

In this case, the ion gel layer 30 forms electric dual layers having a nanometer thickness in an electrode contact surface and has a very high electric capacity of 1 μF/cm² or more, and when the ion gel is applied to the fibrous transistors 100 and 100′ as the gate insulating layer, the same number of charge carriers (electrons or holes), that is, the current, may be obtained at the low driving voltages of the semiconductor layers 12 and 22 or a high current may be obtained at the same operation voltage, so that the device may be driven at the low operation voltage or the high output current may be generated.

For example, the device may be driven at a low operation voltage of 3 V or less, and it is possible to manufacture the fibrous transistors 100 and 100′ having a high device current of 1 mA or more and a current on/off ratio of 10⁵.

It is possible to flexibly dispose the gate 40 by the ion gel layer 30.

The gate 40 is a gate electrode generally used in a transistor, and may serve as a voltage control current source according to an applied voltage. Further, the gate 40 may adjust a flow of the charge carrier of the transistor, and control a current flow between the source and the drain, thereby serving as an amplifier or a switch device.

The gate 40 may be wound in any one form of a spiral form, a parallel form, and a twist form in a longitudinal direction of the ion gel layer 30. Further, the gate 40 may be formed of a conductive material. For example, a metal or a conductive polymer material may be used as the conductive material of the gate 40, and the conductive material may include any one of gold (Au), silver (Ag), aluminum (Al), copper (Cu), nickel (Ni), molybdenum (Mo), tungsten (W), an indium tin oxide (ITO), polythiophene, polyaniline, polyacetylene, olypyrrole, poly phenylene vinylene, a mixture of polyethylenedioxythiophene (PEDOT)/polystyrenesulfonate (PPS), a carbon fiber, and a carbon nano tube (CNT).

However, as long as the gate 40 has conductivity, the material of the gate 40 is not particularly limited.

<Method of Manufacturing Fibrous Transistor>

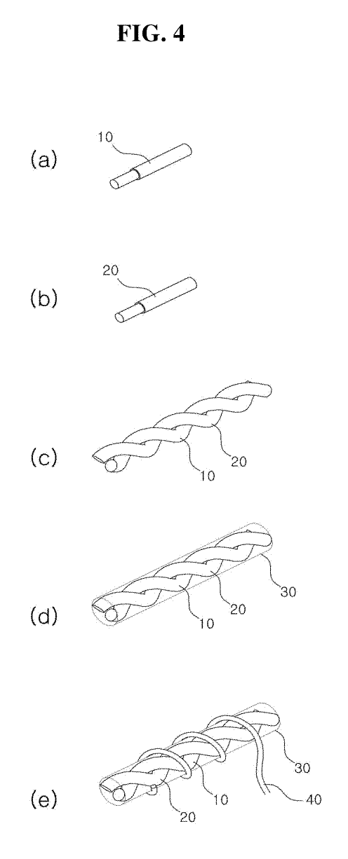

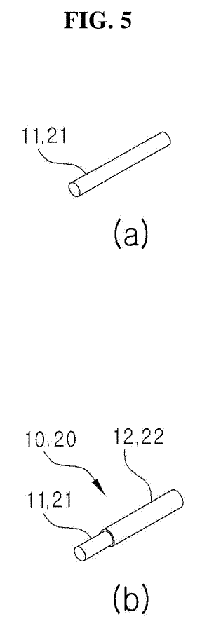

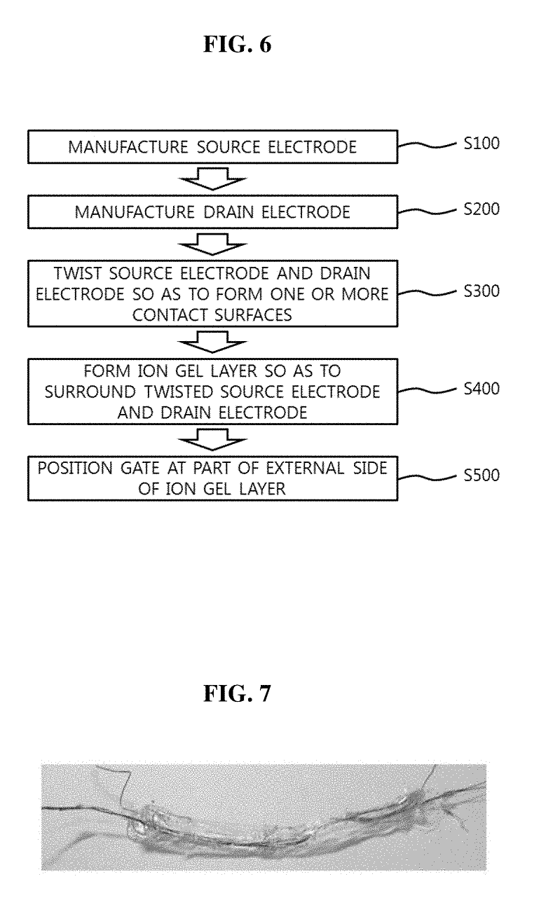

FIG. 4 is a perspective view of a method of manufacturing the fibrous transistors 100 and 100′ according to the exemplary embodiments of the present invention. FIG. 5 is a perspective view illustrating a method of manufacturing the source fiber 10 and the drain fiber 20 according to an exemplary embodiment of the present invention. FIG. 6 is a flowchart of the method of manufacturing the fibrous transistors 100 and 100′ according to the exemplary embodiments of the present invention.

The method of manufacturing the fibrous transistors 100 and 100′ includes (a) an operation of manufacturing the source fiber 10 (S100), (b) an operation of manufacturing the drain fiber 20 (S200), (c) twisting the source fiber 10 and the drain fiber 20 to each other so that the source fiber 10 and the drain fiber 20 form one or more contact surfaces (S300), (d) forming the ion gel layer 30 so as to surround the twisted source fiber 10 and drain fiber 20 (S400), and (e) positioning the gate 40 at a part of an external side of the ion gel layer 30 (S500).

Operations (a) and (b) are operations of preparing the source fiber 10 and the drain fiber 20 including a conductive material, and may further include operations S110 and S210 of preparing conductive fibers 11 and 21, and operations S120 and S220 of forming semiconductor layers 12 and 22 on outer circumferential surfaces of the conductive fibers 11 and 21.

The conductive fibers 11 and 21 are conductive fibers formed of a conductive material, and may supply charges or receive charges. Further, the semiconductor layers 12 and 22 may generally serve as channels in a transistor. The semiconductor layers 12 and 22 are passages, through which the charges generated in the conductive fiber 11 included in the source fiber 10 are moved to the conductive fiber 21 included in the drain fiber 20, and may be formed of a material having charge mobility.

Operation (C) is an operation of making the source fiber 10 and the drain fiber 20 be in contact with each other, and in operation (C), the source fiber 10 and the drain fiber 20 are twisted with each other one or more times to form one or more contact surfaces and form a movement passage of the charges. In this case, the source fiber 10 and the drain fiber 20 may be alternately twisted with each other in a longitudinal direction.

In a longitudinal direction of any one of the source fiber 10 and the drain fiber 20, the other may be wound around the one fiber in a spiral form.

For example, the source fiber 10 serves as a reference fiber, and the drain fiber 20 surrounds the external surface of the source fiber 10 in a spiral form to form one or more contact surfaces. In this case, when the number of times of winding of the drain fiber 20, that is, the number of twists (T/M), is increased, a surface, in which the source fiber 10 is in contact with the drain fiber 20, is increased, and the quantity of current of each of the fibrous transistors 100 and 100′ is increased.

Operation (d) is an operation of forming the ion gel layer 30 so as to surround both the source fiber 10 and the drain fiber 20, and is generally an operation of forming a gate insulating layer in a transistor. In this case, when a co-solvent is used, it is possible to adjust a concentration and viscosity appropriate to printing, so that the ion gel layer 30 may be formed on the external surfaces of the source fiber 10 and the drain fiber 20 by a process, such as aerosol jet printing, ink jet printing, transfer printing, spin coating, dip coating, and casting. Further, the ion gel layer 30 may be formed by a continuous process of a roll-to-roll method.

In the manufacturing process of the fibrous transistors 100 and 100′ using the ionic liquid, a vacuum process is excluded, so that the manufacturing process is relatively simple, and it is possible to manufacture the device through a solution process with low cost, thereby considerably decreasing manufacturing cost.

Operation (e) is an operation of positioning the gate 40 generally serving as a gate electrode in the transistor, and is an operation of positioning the gate 40 only at a part of an external side of the ion gel layer 30 by winding the cylindrical gate 40 around the external side of the ion gel layer 30.

The gate 40 may be formed by being wound around the ion gel layer 30 in a spiral form in a longitudinal direction of the ion gel layer 30. Here, the longitudinal direction of the ion gel layer 30 means the longitudinal direction of the source fiber 10 or the drain fiber 20. That is, the cylindrical gate 40 may be positioned while being wound around the external side of the ion gel layer 30 in the longitudinal direction of the twisted source fiber 10 and drain fiber 20.

The ion gel layer 30 having high ion conductivity and electric capacity surrounds the source fiber 10 and the drain fiber 20, so that when the gate 40 is positioned on the external surface of the ion gel layer 30, the gate 40 may be positioned without a limitation of a shape and a positioned of the gate 40.

<Electromyogram Sensor>

An electromyogram sensor may include one or more transistors manufactured by the method of manufacturing the transistor. The electromyogram sensor may be attached to a body of a user and serve to measure an electromyogram signal. The electromyogram is a record of an electric activity of muscle, and may record an electric activity of muscle by using an extracellular electrode and obtain information about a state of skeletal muscles or innervation for skeletal muscles. A normally innervated muscle does not represent an electric activity in a stable state, and an electromyogram signal used in a clinical field is obtained by measuring activity potential represented between a recording electrode installed around a contracted muscular fiber and a reference electrode, which is spaced apart from the recording electrode by a predetermined distance. The electromyogram sensor measuring the electromyogram is also referred to as a bioelectrode, and is released in two types of a plane electrode and a needle electrode.

The electromyogram sensor according to the present invention may be positioned so that a gate electrode positioned at an outer circumferential portion of the transistor is in contact with the skin. Further, a voltage of the gate electrode is changed by the electromyogram generated when the muscle is contracted, and a current value of a drain is changed, so that the electromyogram sensor measures electromyogram.

<Electrocardiogram Sensor>

An electrocardiogram sensor may include one or more transistors manufactured by the transistor manufacturing method. Electrocardiogram is a diagram recording an action current and an action potential difference according to a contraction of the heart with a wavy line, and may be generally represented with several electrocardiographic curves by using an electrocardiograph.

The electrocardiogram sensor according to the present invention may be positioned so that a gate electrode positioned at an exterior portion of the transistor is in contact with a skin. Further, the electrocardiogram sensor measures a potential difference generated according to the contraction and the relaxation of the heart and measures an electrical activity of the heart.

<Manufacturing Example>

FIG. 7 is a picture of a fibrous transistor according to a manufacturing example.

Here, a source fiber and a drain fiber are conductive fibers, in which a poly(3-hexylthiophene) (P3HT) semiconductor layer is formed in a conductive fiber formed of a conductive polymer poly(3,4-ethylenedioxythiophene) doped with poly(4-styrenesulffonate), and were twisted in a longitudinal direction for preparation. Then, after the twisted source fiber and drain fiber were inserted into a disposable spuit pipe, an ion gel was filled inside an internal empty space of the spuit pipe by using a syringe, so that an ion gel layer was formed on outer circumferential surfaces of the source fiber and the drain fiber. Further, the disposable spuit pipe was removed, and the prepared copper fiber was positioned at an external side of the ion gel layer to manufacture a fibrous transistor.

### Experimental Example

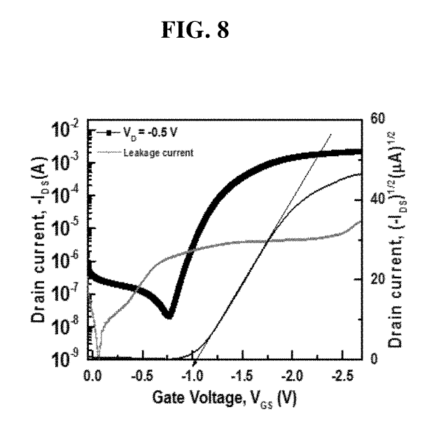

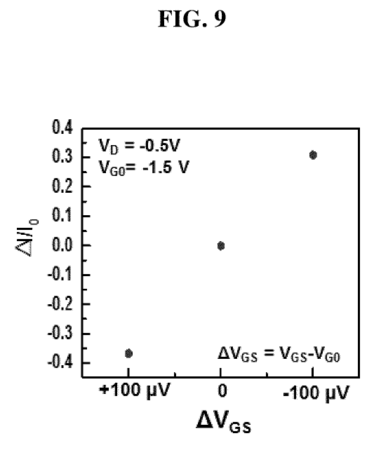

A driving performance measurement experiment of the fibrous transistor manufactured by the manufacturing example was conducted. FIG. 8 is a result of a driving performance measurement experiment of the fibrous transistor, and FIG. 9 is a graph representing a change in a drain current according to a change in a gate voltage of the fibrous transistor according to the present invention.

Referring to FIG. 8, an experiment for a change in a current between the source and drain fibers when a predetermined voltage of −0.5 V was applied to the drain fiber and a voltage applied to the gate was increased was performed. As a result, when a voltage applied to the gate is about 1.5 V, a current between the source fiber and the drain fiber is sharply increased, and in this case, the fibrous transistor becomes an on-state. Further, charge mobility is determined according to a speed of a change from on to off of the fibrous transistor by decreasing a voltage applied to the gate.

The present invention has been described with reference to the exemplary embodiments, but those skilled in the art may understand that the present invention may be variously modified and changed within the scope without departing from the spirit and the area of the present invention described in the accompanying claims.

Referring to FIG. 9, there was conducted an experiment for a change in the quantity of current of the drain fiber when a predetermined voltage of −0.5 V is applied to the drain fiber and a predetermined voltage of −1.5 V is applied to the gate, and the voltage applied to the gate is changed within the range of −100 μV to 100 μV. As a result, it can be seen that when the voltage applied to the gate is small, the change in the current of the drain fiber is increased.

The present invention has been described with reference to the exemplary embodiments, but those skilled in the art may understand that the present invention may be variously modified and changed within the scope without departing from the spirit and the area of the present invention described in the accompanying claims.

What is claimed is:

1. A fibrous transistor, comprising:
a source fiber;
a drain fiber;
an ion gel layer provided so as to surround the source fiber and the drain fiber; and
a gate positioned at a part of an external side of the ion gel layer,
wherein the source fiber and the drain fiber form one or more contact surfaces in a twisted state.

2. The fibrous transistor of claim 1, wherein the source fiber includes a conductive fiber, and a semiconductor layer formed on an outer circumferential surface of the conductive fiber.

3. The fibrous transistor of claim 1, wherein the drain fiber includes a conductive fiber, and a semiconductor layer formed on an outer circumferential surface of the conductive fiber.

4. The fibrous transistor of claim 1, wherein the source fiber and the drain fiber are alternately twisted with each other in a longitudinal direction.

5. The fibrous transistor of claim 1, wherein in a longitudinal direction of any one fiber between the source fiber and the drain fiber, the other fiber is wound around the one fiber in a spiral form.

6. The fibrous transistor of claim 1, wherein the gate is wound in a spiral form in a longitudinal direction of the ion gel layer.

7. The fibrous transistor of claim 2, wherein the conductive fiber includes a conductive material.

8. The fibrous transistor of claim 2, wherein the semiconductor layer includes one or more of an organic semiconductor, an oxide semiconductor, a metal oxide semiconductor, and a carbon compound semiconductor.

9. The fibrous transistor of claim 1, wherein the ion gel layer includes at least one of 1-ethyl-3-methylimidazolium bis(trifluoromethylsulfonyl)imide ([EMIM][TFSI]), poly(vinyl phosphonic acid-co-acrylic acid) (P(VPA-AA)), poly(styrene sulfonic acid) (PSSH), NaCl among the poly ethylene oxide (PEO) matrixes, polyvinylidenefluoride (PVDF), 1-butyl-3-methylimidazolium bis (trifluoromethanesulfonimide) ([bmim][Tf2N]), polymer IL poly(1-vinyl-3-methylimidazolium bis(tri-fluoromethanesulfonimide) (poly[ViEtIm][Tf2N]), PEO/LiTFSI, 1-butyl-3-methylimidazolium hexafluorophosphate ([BMIM][PF6]), and 1-ethyl-3-methylimidazolium n-octylsulfate ([EMIM][OctOSO3]).

10. A method of manufacturing a fibrous transistor, the method comprising:
(a) manufacturing a source fiber;
(b) manufacturing a drain fiber;
(c) twisting the source fiber and the drain fiber so as to form one or more contact surfaces;
(d) forming an ion gel layer so as to surround the twisted source fiber and drain fiber; and
(e) positioning a gate at a part of an external side of the ion gel layer.

11. The method of claim 10, wherein operations (a) and (b) further include:
preparing a conductive fiber; and
forming a semiconductor layer on an outer circumferential surface of the conductive fiber.

12. The method of claim 10, wherein in operation (c), the source fiber and the drain fiber are alternately twisted with each other in a longitudinal direction.

13. The method of claim 10, wherein in operation (c), in a longitudinal direction of any one fiber between the source fiber and the drain fiber, the other fiber is wound around the one fiber in a spiral form.

14. The method of claim 10, wherein in operation (d), the ion gel layer is formed by any one method of aerosol jet printing, ink jet printing, transfer printing, spin coating, dip coating, and casting.

15. The method of claim 10, wherein in operation (e), the gate is positioned in any one form among a spiral form, a parallel form, and a twist form in a longitudinal direction of the ion gel layer.

16. An electromyogram sensor comprising a fibrous transistor manufactured by claim 10.

17. An electrocardiogram sensor comprising a fibrous transistor manufactured by claim 10.
